(19) United States
(12) Patent Application Publication
(10) Pub. No.: US 2016/0339057 A1
(43) Pub. Date: Nov. 24, 2016

(54) NOVEL COMPOSITION METHOD OF USING THE SAME FOR THE TREATMENT OF LYME DISEASE

(71) Applicants: MATTHIAS W. RATH, APTOS, CA (US); Aleksandra Niedzwiecki, APTOS, CA (US); Anna Goc, SANJOSE, CA (US)

(72) Inventors: MATTHIAS W. RATH, APTOS, CA (US); Aleksandra Niedzwiecki, APTOS, CA (US); Anna Goc, SANJOSE, CA (US)

(73) Assignee: MATTHIAS W. RATH, APTOS, CA (US)

(21) Appl. No.: 15/229,245

(22) Filed: Aug. 5, 2016

Related U.S. Application Data
(63) Continuation of application No. 14/517,887, filed on Oct. 19, 2014.

(51) Int. Cl. A61K 33/18; A61K 31/231; A61K 31/375; A61K 31/216; A61K 31/352; A61K 36/87; A61K 31/593; A61K 31/22

(52) U.S. Cl. CPC A61K 33/18; A61K 31/593; A61K 31/231; A61K 31/22; A61K 31/216; A61K 31/352; A61K 36/87; A61K 31/375

(57) ABSTRACT

Lyme disease caused by B. burgdorferi and B. garinii has been traditionally treated with antibiotics. However, to eradicate all three morphological forms of Borrelia species such as spirochetes, rounded bodies and biofilm more effective and safe treatment plan is required. In the instant application, Vitamins and Phytobiologicals have been tested for as a short term and long term treatment in vitro and on biofilm. The combination of tested compounds in form of Mix AO seems to be very beneficial in treating Lyme disease caused by B. burgdorferi and B. garinii.

### CROSS REFERENCE TO RELATED APPLICATION

This application is a continuation of pending application U.S. Ser. No. 14/517,887, filed on 19 Oct. 2014. The pending U.S. applications is hereby incorporated by reference in its entireties for all of its teachings.

### FIELD OF TECHNOLOGY

This disclosure relates generally to a novel composition and the use of novel composition to treat Lyme disease.

### BACKGROUND

Lyme disease is the most common tick-borne illness in the world today. Borrelia burgdorferi and Borrelia garinii are bacteria transmitted by ticks and cause Lyme disease. Borrelia garinii has only been found in ticks in Eurasia, while Borrelia burgdorferi is found in North America and Eurasia. The Center for Disease Control (CDC) states that there may be more than 300000 cases and as high as one million cases per year in United States alone (Stricker et al. 2014). Stricker (2014) further states that Lyme disease is approaching an epidemic proportion and the monotherapy of using antibiotics may not be sufficient. Stricher et al. (2011) states that chronic Lyme disease involves latent forms such as rounded bodies and biofilm formation. This publication further emphasizes that there is a need to develop new drugs to target these novel infectious processes. Development of agents other than antibiotics that would not cause bacterial resistance, but effectively target spirochetes, rounded forms and biofilms may also provide valuable insight into the treatment of other chronic infections, not only Lyme disease. There is a need to find more effective drugs for use as a treatment of Lyme disease.

### SUMMARY

The current invention discloses a method of making and using a novel composition of vitamins and other plant-derived bioactive compounds (phytobiologicals) and using the same to treat Lyme disease. In one embodiment, several compounds in permutation and combination were made and tested for bactericidal and bacteriostatic effect against Borrelia burgdorferi and Borrelia garinii. In another embodiment, the individual compounds were, but not limited to, Vitamin D3, Vitamin C, L-lysin, Tranexamic acid, Quercetin 3D, Hydroxytyrosol, Fulvic acid, Teasel Root Extract, Cis-2 decenoic acid, Serrapeptase, Trimesic acid, Aminocaproic acid, Defferoxamine, Ellagic acid, Oregano oil, Oleuropein, Apigenin, Luteolin, Kelp (Kelp (Iodine)), Rottlerin, Grape seed extract (OPC), Malvidin, Piceatannol, Aronia, Myricetin, Rosmarinic acid, Kaempherol, Baicalein, Monolaurin, E-viniferin, Olein, Fucoinad, Nordihydroguaiaretic acid, Morin, Fisetin and Vitamin B complex.

In one embodiment, several Mixes were made and tested to find the optimum Mix having the best bactericidal and bacteriostatic effect on specific species of bacteria that caused Lyme disease. The Mixes were as following:

Mix A: Vitamin D3, Cis-2-decenoic acid, Kelp (Iodine), Monolaurin, Luteolin, Rosmarinic acid.
Mix A1: Vitamin D3, Cis-2-decenoic acid, Kelp (Iodine), Monolaurin, Serrapeptase, Luteolin, Rosmarinic acid.

### Mix B: Hydroxytyroslo, Morin, Oenin, E-Viniferin, Baicalein.

Mix C: Vitamin D3, Cis-2-decenoic acid, Kelp (Iodine), Monolaurin, Serrapeptase, Luteolin, Rosmarinic acid, Hydroxytyrosol, Morin, Oenin, E-Viniferin, Baicalein.
Mix D: Vitamin D3, Cis-2-decenoic acid, Kelp (Iodine), Monolaurin, Luteolin, Hydroxytyrosol.
Mix E: Vitamin D3, Vitamin C, Cis-2-decenoic acid, Kelp (Iodine), Monolaurin, Luteolin, Hydroxytyrosol.
Mix F: Vitamin D3, Cis-2-decenoic acid, Kelp (Iodine), Monolaurin, Luteolin, Hydroxytyrosol, Rosmarinic acid.
Mix G: Vitamin D3, Vitamin C, Cis-2-decenoic acid, Kelp (Iodine), Monolaurin, Luteolin, Hydroxytyrosol, Rosmarinic acid.
Mix H: Vitamin D3, Vitamin C, Cis-2-decenoic acid, Kelp (Iodine), Monolaurin, Luteolin, Hydroxytyrosol, Rosmarinic acid, Baicalein.
Mix I: Vitamin D3, Vitamin C, Cis-2-decenoic acid, Kelp (Iodine), Monolaurin, Luteolin, Hydroxytyrosol, Rosmarinic acid, Baicalein, Oenin.
Mix J: Vitamin B complex, Vitamin C, Cis-2-decenoic acid, Monolaurin.
Mix K: Vitamin B complex, Vitamin C, Kelp (Iodine), Monolaurin, Luteolin.
Mix L: Vitamin B complex, Vitamin C, Kelp (Iodine), Monolaurin, Oenin.
Mix M: Vitamin B complex, Vitamin C, Kelp (Iodine), Monolaurin, Rosmarinic acid.
Mix N: Vitamin B complex, Vitamin C, Kelp (Iodine), Monolaurin, Baicalein.
Mix O: Vitamin B complex, Vitamin C, Kelp (Iodine), Monolaurin, Rosmarinic acid, Baicalein.
Mix P: Vitamin B complex, Vitamin C, Hydroxytyrosol, Monolaurin, Rosmarinic acid, Baicalein, Luteolin.
Mix AO: Vitamin D3, Cis-2-decenoic acid, Kelp (Iodine), Monolaurin, Luteolin, Rosmarinic acid, Vitamin B complex, Vitamin C and Baicalein.

In one embodiment, bacteria that cause Lyme disease were selected. The selected species were Borrelia burgdorferi and Borrelia garinii. In another embodiment, these bacteria were grown individually as a biofilms to test the efficacy of the Mix A-P. In another embodiment, series of studies were performed to test the efficacy of the Mixes for bactericidal and bacteriostatic effects at their different concentrations.

In one embodiment, the best Mixes were used over a period of time to determine as a repeated treatment method in a short and long term resistance study for these Mixes to prevent the growth of the bacteria.

In one embodiment, three morphological forms of B. burgdorferi and B. garinii (spirochetes, rounded bodies, and biofilm) were subjected to testing and analysis. Since Borrelia sp. are aero-tolerant anaerobes thus they were cultured stationary in the presence of 5% CO₂ in tightly screw-caped tubes.

In another embodiment, the first set of experiments was focused on establishing what compound/mixture will inhibit growth of B. burgdorferi and B. garinii. In another embodiment, a set of experiments was intended to check what compound/mixture will express the bactericidal effect on B. burgdorferi and B. garinii. In another embodiment, a set of experiments was design to evaluate what compound/mixture will reveal bacteriostatic and/or bactericidal effect against biofilms of B. burgdorferi and B. garinii. In another embodiment, a set of experiments was performed to establish the minimal concentration of compound/mixture that expresses the bacteriostatic and/or bactericidal effect on B. burgdorferi and B. garinii. As another embodiment, a set of experiments was carried out to check whether tested mixtures and at what concentrations will cause B. burgdorferi and B. garinii to become resistant after short and long-period of undergone treatment.

In one embodiment, a novel composition is being proposed for the treatment of Lyme disease. A treatment method with a novel compound for treating all three forms of the bacteria (once infected is found in the blood stream) is being treated simultaneously in the cells. In one embodiment, a superior treatment effect is observed either alone or in conjunction with regular antibiotic is shown. In one embodiment, a kit and a pharmaceutical composition is also disclosed with Mix A to P, AO.

In one embodiment, a method, comprises of making a mixture at a specific concentration using at least one of a vitamin, a small chain fatty acid signaling molecule, an essential amino acid, a Lauric acid derivative, a flavonoid, a phenol, a plant extract, and a mineral; and treating of a Lyme disease caused by a specific bacteria using the mixture and preventing progression of the disease from the acute stage to the chronic stage, wherein the preventing progression of the Lyme disease is the advancement of the Lyme disease from spirochete stage to a rounded bodies and further to the formation of a biofilm.

The composition, method, and treatment disclosed herein may be implemented in any means for achieving various aspects, and may be executed in a form suitable for the mammal.

### BRIEF DESCRIPTION OF DRAWINGS

Example embodiments are illustrated by way of example and not limitation in the Figures of the accompanying drawings, in which like references indicate similar elements and in which:

FIG. 1 A shows susceptibility of the spirochetes of B. burgdorferi (left panel) and FIG. 1 B B. garinii (right panel) to the most effective concentration of the chosen three most effective mixtures.

FIG. 2 A shows susceptibility of the spirochetes of B. burgdorferi (left panel) and FIG. 2 B B. garinii (right panel) to doxycycline.

FIG. 3A shows susceptibility of the rounded forms of B. burgdorferi (left panel) and FIG. 3B B. garinii (right panel) to the most effective concentration of the chosen three most effective mixtures.

FIG. 4 A shows susceptibility of the rounded forms of B. burgdorferi (upper panel) and FIG. 4 B B. garinii (lower panel) to doxycycline.

FIGS. 5 A, B, C, D and E shows analysis of two Borrelia species on biofilm and their treatment effect with Mix's as therapy.

FIGS. 6 A, B, C, D, and E shows analysis of two Borrelia species on biofilm and their treatment effect with Mixes's as prevention.

FIGS. 7 A and B shows analysis of two Borrelia species on biofilm and their treatment effect with doxycycline.

FIGS. 8 A, B, C, D, E and F shows analysis of spirochetes of two Borrelia species treated with different concentrations of the chosen three most effective mixtures.

FIGS. 9 A, B and C shows susceptibility of the rounded forms of B. burgdorferi to the different concentrations of the chosen three most effective mixtures.

FIGS. 10 A, B and C shows susceptibility of the rounded forms of B. garinii to the different concentrations of the chosen three most effective mixtures.

FIGS. 11 A, B, C,D, E and F shows susceptibility of the spirochetes of B. burgdorferi (left panel) and B. garinii (right panel) treated with different concentrations of the chosen three most effective mixtures for a short term resistance.

FIGS. 12 A, B and C shows susceptibility of the rounded forms of B. burgdorferi treated with different concentrations of the chosen three most effective mixtures for a short term resistance.

FIGS. 13 A, B and C shows susceptibility of the rounded forms of B. garinii to the different concentrations of the chosen three most effective mixtures for a short term resistance.

FIGS. 14 A, B, C,D, E and F shows susceptibility of the spirochetes of B. burgdorferi (left panel) and B. garinii (right panel) treated with different concentrations of the chosen three most effective mixtures for a long term resistance.

FIGS. 15 A, B and C shows susceptibility of the rounded forms of B. burgdorferi treated with different concentrations for a long term resistance.

FIGS. 16 A, B and C shows susceptibility of the rounded forms of B. garinii to the different concentrations of the chosen three most effective mixtures at different concentration for a long term resistance.

Other features of the present embodiments will be apparent from the accompanying drawings and from the detailed description that follows.

### DETAILED DESCRIPTION

In the instant disclosure a novel composition of natural and synthetic compounds were tested and used for bactericidal and bacteriostatic effect on three morphological forms of B. burgdorferi and B. garinii (spirochetes, rounded bodies, and biofilm) as a treatment for Lyme disease. Since Borrelia sp. are aero-tolerant anaerobes thus they were cultured stationary in the presence of 5% CO₂ in tightly screw-caped tubes. The first set of experiments was focused on establishing what compound/mixture will inhibit growth of B. burgdorferi and B. garinii. Second set of experiments was intended to check what compound/mixture will express the bactericidal effect on B. burgdorferi and B. garinii. The third set of experiments was design to evaluate what compound/mixture will reveal bacteriostatic and/or bactericidal effect against biofilms of B. burgdorferi and B. garinii. The fourth set of experiments was performed to establish the minimal concentration of compound/mixture that expresses the bacteriostatic and/or bactericidal effect on B. burgdorferi and B. garinii. The last set of experiments was carried out to check whether tested mixtures and at what concentrations will cause B. burgdorferi and B. garinii to become resistant after short and long-period of undergone treatment. The accompanying tables and figures show the results of various sets of experiments and prove that the composition and the method of treatment is very effective in short and long term for all forms of B. burgdorferi and B. garinii.

### Materials and Methods

Test Compounds:

The following compounds, with the purity between 90%-98% according to the manufacturer, were obtained from Sigma (St. Louis, Mo.): Vitamin D3, Vitamin C, L-Lysin, Tranexamic acid, Quercetin 3D, Deferoxamine, Fucoidan, ε-Viniferin, Ellagic acid, Hydroxytyrosol, Baicalein, cis-2-decenoic acid, Morin, Oenin, Oleuropein, Nordihydroguaiaretic acid, Myricetin, Malvidin, and as a positive control antibiotic doxycycline (100 mg/ml stock suspension in absolute ethanol) since it is use as a standard therapeutic treatment for patients with Lyme disease. The following compounds, with the purity between 97%-99% according to the manufacturer, were purchased from Tocris Bioscience (Bristol, United Kingdom): Rosmarinic acid, Kaempferol, Piceatannol, Rottlerin, Grape seed extract (OPC), Luteolin, Fisetin, Apigenin. Other reagents used in this study were: organic kelp with standardized Kelp (Iodine) content (i.e. 150 μg/ml as a 100 of % Daily Value) purchased from World Organic Ltd., New Zealand), monolaurin (Lauricidin®) bought from Med-Chem Laboratories, Inc., Goodyear, Ariz. as a pure sn-1 monolaurin (glycerol monolaurate) derived from coconut oil, serrapeptase (SerraEzyme 80,000 Iu™) obtained from Good Health Naturally, USA with unspecified by manufacture's purity, vitamin B-complex “100” purchased from Solgar, Inc., NJ with unspecified by manufacture's purity, fulvic acid from Nano Health Solution Inc. Charlston N.C. with unspecified by manufacture's purity, organic teasel root extract in 45-55% of ethanol from Woodland Essence, Cold Brook, N.Y. with unspecified by manufacture's purity, aminocaprionic acid from Selleckchem.com Houston Tex. with unspecified by manufacture's purity, and oregano oil (with standardized i.e. 70% of carvacrol content) from VitaCost, Lexington, N.C. with unspecified by manufacture's purity.

Preparation of Test Compounds for Susceptibility Testing.

A stock solution of 50-100 mg/ml for solid compounds (depending on solubility of each substance) was prepared by suspending each of the test compounds in absolute ethanol. All stock solutions were stored in aluminum foil-wrapped tubes at −20° C. Since a high percentage of ethanol could be bactericidal, the amount of ethanol added to the growth medium was kept as low as possible in order to minimize the potential effect on growth of Borrelia sp. A preliminary experiment was carried out to determine the maximum percentage of ethanol which could be applied without growth inhibition of Borrelia sp. and was found to be 0.5% (vol/vol) (data not shown). The final concentration of ethanol present in the growth medium was kept below 0.4% (vol/vol). Each stock solution was serially diluted in 10% ethanol, and appropriate amount of each dilution was then added to 1.5 ml sterile screw-cap test tubes containing 1 ml of BSH complete medium to yield final concentrations of 100-1000 μg/ml for solid compounds. For doxycycline, as a positive control, the final used concentration was 250 μg/ml. As a negative control ethanol at 0.1-0.4% (vol/vol) was applied.

Test Microorganisms.

Two Borrelia species i.e. Borrelia burgdorferi and Borrelia garinii were tested in this study. Low passage isolates of the B31 strain of B. burgdorferi and CIP103362 strain of B. garinii were obtained from the American Type Culture Collection (Manassas, Va.). The stocks of both species were cultured in commonly used conditions, i.e. medium such as Barbour-Stoner-Kelly H (BSK-H) supplemented with 6% rabbit serum (Sigma, St Louis, Mo.) without antibiotics at 33° C. with 5% CO₂, in sterile screw-cap 15 mL polypropylene tubes with or without gentile shaking depends on type of experiment. B31 strain is an isolate from Ixodes dammini whereas CIP103362 strain is an isolate from Ixodes ricinus. Both strains are well known human pathogenic factors of Lyme disease. So far, Borrelia burgdoferi has been found in ticks from North America and Eurasia, while Borrelia garinii only in ticks in Eurasia.

Preparation of Test Microorganisms for Susceptibility Testing.

The strains of B. burgdorferi and B. garinii were prepared for testing as described by Sapi et al. Infection and Drug Resistance 2011. Briefly, the strains were activated from original cryobank vials and inoculated into 10 ml BKS-H compete medium, and maintained at 33° C. For generation of homogeneous cultures (i.e. having only spirochete form) of B. burgdorferi or B. garinii, spirochetes were inoculated and maintained in a shaking incubator at 33° C. and 250 rpm, where there is no biofilm formation (Sapi et al. Infection and Drug Resistance 2011 supported by own observation). For generation of biofilm-like colonies of B. burgdorferi or B. garinii, spirochetes were inoculated in four-well chambers (BD Biosciences, Sparks, Md.) coated with rat-tail collagen type I and incubated for 1 week without shaking.

Bacteriostatic Assessment of Test Compounds on Test Microorganisms.

To determine what compound/mixture will inhibit visible growth of B. burgdorferi and B. garinii, a new and accepted micro-dilution method was used according to Sapi et al. Infection and Drug Resistance 2011. Briefly, sterile 1.5 ml test tightly caps screwed tubes containing 1 ml BSK-H medium, supplemented with the test compound of interest were inoculated with 2×10⁶ CFU/ml of the homogenous bacterial suspension. The tubes were then incubated at 33° C. and growth was monitored at regular intervals for up to 72 h. The whole experiment was repeated three times for each strain and each concentration. Control cultures were treated with ethanol (i.e. 0.1-0.4 vol/vol) alone. For doxycycline, the final used concentration was 250 μg/m1. Cell growth was assessed by a bacterial Petroff-Hausser counting chamber after the 0 hour, 24 hrs, 48 hrs, 72 hrs and 7 days of incubation using dark field microscopy (direct cell counting), as a standard procedure.

Evaluation of Bactericidal Effect of Test Compounds on Test Microorganisms.

To determine what compound/mixture will have the bactericidal effect a fluorescence method was used according to Sapi et al. Infection and Drug Resistance 2011. Briefly, 2×10⁶ CFU/ml of the homogenous bacterial suspension was inoculated into each sterile 1.5 ml test tightly caps screwed tubes containing 1 ml BSK-H medium, supplemented with the test compound/mixture of interest. Control cultures were treated with ethanol (i.e. 0.1-0.4 vol/vol) alone. For doxycycline, as a positive control, the final used concentration was 250 μg/m1. The tubes were then incubated at 33° C. and viability was monitored at regular intervals for up to 72 h. The whole experiment was repeated three times for each strain and each concentration. The susceptibility of spirochetes and round body forms to the test compound was then assessed after 24 h, 48 h, 72 h and 7 days by LIVE/DEAD® BacLight™ Bacterial Viability Assay using fluorescent microscopy, were the ratio of live (green) and dead (red) B. burgdorferi and B. garinii morphological forms were calculated.

Valuation of Test Compounds on Bacterial Biofilm.

Qualitative effect of the test compounds against biofilm-like colonies of B. burgdorferi and B. garinii was evaluated using commonly used and well accepted crystal violet (CV) staining method, according to protocol provided by Sapi et al. Infection and Drug Resistance 2011. Briefly, for curable effect, 1×10⁷ CFU/ml from homogeneous cultures of spirochetes were inoculated in extracellular matrix proteins (i.e. matrigel, collagen type I, fibrinogen, hyaluronan, and chondroitin sulfate) coated four-well chambers and incubated for 1 week, respectively. After the 1 week of incubation, biofilm-like colonies were treated with various compounds/mixtures. For preventive effect, 1×107 CFU/ml from homogeneous cultures of spirochetes complemented with various compounds/mixtures were inoculated in extracellular matrix proteins (i.e. matrigel, collagen type I, fibrinogen, hyaluronan, and chondroitin sulfate) coated four-well chambers and incubated for 1 week, respectively. Control wells were treated with ethanol (i.e. 0.1-0.4 vol/vol) alone. For doxycycline, as a positive control, the final used concentration was 250 m/ml. The chambers were then incubated at 33° C. for 72 h. The whole experiment was repeated three times for each strain and each concentration. Next, all wells were fixed with 500 μl of cold methanol-formalin (1:1) for 30 minutes and stained with 1 ml of crystal violet (0.1%) for 10 minutes. The biofilm-like colonies were carefully washed three times with 1×PBS and 1 ml of methanol was added to each well to extract a dye, which was measured at 595 nm wavelength.

Micro-Dilution Study.

To determine what concentration of compound/mixture will express the bacteriostatic and/or bactericidal effect, sterile 1.5 ml test tightly caps screwed tubes containing 1 ml BSK-H medium, supplemented with the test compound/mixture of interest diluted accordingly (i.e. 5×, 10×, 20×, and 50×) were inoculated with 2×10⁶ CFU/ml of the homogenous bacterial suspension. The tubes were then incubated at 33° C. and growth was monitored at regular intervals for up to 7 days. The whole experiment was repeated three times for each strain and each concentration. Control cultures were treated with ethanol (i.e. 0.1-0.4 vol/vol) alone. Cell growth was assessed by a bacterial Petroff-Hausser counting chamber after the 24, 48, and 72-hour incubation using dark field microscopy (direct cell counting), as a standard procedure, whereas bactericidal effect was assessed after by LIVE/DEAD® BacLight™ Bacterial Viability Assay using fluorescent microscopy, were the ratio of live (green) and dead (red) B. burgdorferi and B. garinii morphological forms were calculated.

The Mix A, O and AO were selected for further studies. The composition of the Mixes were as follows:

Mix A: Vitamin D3—0.5-1.5 nM, Cis-2-decenoic acid—150-300 μg/ml, Kelp (Iodine)—1-10 μg/ml, Monolaurin—500-1500 μg/ml, Luteolin—50-150 μg/ml, Rosmarinic acid—200-300 μg/ml. Mix O: a Vitamin B complex—0.5-1.5 mg/ml, Vitamin C—0.1-0.3 mM, Kelp (Iodine)—1-10 μg/ml, Monolaurin—500-1500 μg/ml, Rosmarinic acid—200-300 μg/ml, Baicalein—500-1500 μg/ml. Mix AO: wherein the mixture is a Mix AO, wherein the Mix AO consists of a Vitamin D3—0.5 nM-1.5 nM, Cis-2-decenoic acid—200-300 μg/ml, Kelp (Iodine) 1-10 μg/ml, Monolaurin—500-1500 μg/ml, Luteolin—50-150 μg/ml, Rosmarinic acid—150-300 μg/ml, Vitamin B complex—0.5-2.0 mg/ml, Vitamin C—0.1-0.3 mM, Baicalein—500-1500 μg/ml. The test was conducted for 1×, 5×, 10×, 20× and 50× for the two bacteria and biofilm. The times that were tested were for 0 hour, 24 hours, 48 hours, 72 hours and 7 days. Doxycycline was also tested along with the said Mixes. The curative and preventive effect for each combination was also tried. Throughout this specification capital and small letters are used for different components of the Mixes and they mean the same.

Resistance Study.

To check what concentration of compound/mixture will cause Borrelia sp. resistant, the bacteriostatic and/or bactericidal effect was determined. Briefly, sterile 1.5 ml test tightly caps screwed tubes containing 1 ml BSK-H medium, supplemented with the test compound/mixture of interest diluted accordingly (i.e. 5×, 10×, 20×, and 50×) were inoculated with 2×10⁶ CFU/ml of the homogenous bacterial suspension. The tubes were then incubated at 33° C. and growth was monitored at regular intervals for up to 7 days followed recovery period by inoculating 100 μl of bacterial suspension to fresh 1 ml BSK-H medium w/o test compound/mixture of interest for 5-7 days and again inoculation to the sterile 1.5 ml test tightly caps screwed tubes containing 1 ml BSK-H medium, supplemented with the test compound/mixture of interest diluted accordingly (i.e. 5×, 10×, 20×, and 50×) were inoculated with 2×10⁶ CFU/ml of the bacterial suspension. For short-term resistance study, this cycle was repeated 4 times that ended up with final treatment of undiluted test compound/mixture of interest. For long-term resistance study, this cycle was also repeated 4 times followed next 15 passages, each in fresh 1 ml BSK-H medium w/o treatment with test compound/mixture of interest, that ended up with the final treatment of undiluted test compound/mixture of interest. The whole experiment was repeated three times for each strain and each concentration. Control cultures were treated with ethanol (i.e. 0.1-0.4 vol/vol) alone. Cell growth was assessed by a bacterial Petroff-Hausser counting chamber after the 24, 48, 72 h, and 7 days of incubation using dark field microscopy (direct cell counting), as a standard procedure, whereas bactericidal effect was assessed after by LIVE/DEAD® BacLight™ Bacterial Viability Assay using fluorescent microscopy, were the ratio of live (green) and dead (red) B. burgdorferi and B. garinii morphological forms were calculated.

Statistical Analysis.

Means and standard deviations were determined for all experiments and Student's t test analysis was used to determine significant differences. Statistical analysis was performed by two-sample paired t-test using GraphPad statistical software.

### Results and Discussion

Several experiments were conducted and systematic approach was taken to test all the mixtures in different time frames, different Borrelia sp., different bacterial morphological forms, and different composition of the compounds in the medium. The first set of experiments was focused on establishing what compound/mixture will inhibit growth of B. burgdorferi and B. garinii over a period of time. Table 1-3 shows all the Mixes from A1 to P being tested.

TABLE 1
Various mixtures tested for 24 hour and 48 hour time frames.

| Tested | % ± SD of control after 24 hrs | | | % ± SD of control after 48 hrs | | |
|---|---|---|---|---|---|---|
| mix | SP | RB | dead RB | SP | RB | dead RB |
| Mix A1: | 58.6 ± 8.1 | 323.4 ± 67.4 | 29.9 ± 4.1 | 50.0 ± 8.1 | 322.5 ± 18.5 | 29.9 ± 4.3 |
| Mix B: | 51.7 ± 6.7 | 452.9 ± 35.5 | 42.8 ± 4.2 | 44.1 ± 6.7 | 357.5 ± 19.6 | 35.4 ± 3.5 |
| Mix C: | 27.6 ± 2.9 | 405.9 ± 9.6 | 32.7 ± 4.3 | 7.8 ± 3.0 | 468.9 ± 10.1 | 33.7 ± 3.4 |
| Mix D: | 20.5 ± 5.8 | 372.7 ± 29.9 | 20.8 ± 2.9 | 22.8 ± 2.7 | 330.3 ± 17.6 | 20.2 ± 2.2 |
| Mix E: | 15.4 ± 2.7 | 348.5 ± 37.8 | 32.2 ± 4.3 | 17.9 ± 2.4 | 315.2 ± 24.4 | 34.2 ± 4.3 |
| Mix F: | 11.5 ± 2.9 | 333.3 ± 37.8 | 20.4 ± 3.5 | 24.4 ± 3.3 | 296.9 ± 14.1 | 25.1 ± 4.5 |
| Mix G: | 19.0 ± 2.4 | 351.5 ± 22.9 | 24.2 ± 8.4 | 22.8 ± 3.3 | 245.5 ± 18.8 | 30.0 ± 7.3 |
| Mix H: | 18.8 ± 3.9 | 344.4 ± 26.7 | 42.1 ± 5.3 | 22.2 ± 2.4 | 317.3 ± 23.8 | 38.1 ± 3.0 |
| Mix I: | 16.7 ± 3.1 | 311.1 ± 24.4 | 41.5 ± 2.3 | 12.1 ± 5.7 | 334.6 ± 20.0 | 32.9 ± 4.0 |
| Mix J: | 25.0 ± 3.0 | 211.3 ± 11.6 | 27.9 ± 3.5 | 40.0 ± 3.0 | 256.5 ± 17.3 | 27.8 ± 6.8 |
| Mix K: | 21.4 ± 6.0 | 253.5 ± 20.8 | 29.9 ± 3.3 | 40.5 ± 7.0 | 278.2 ± 29.9 | 29.8 ± 6.7 |
| Mix L: | 28.6 ± 4.0 | 215.9 ± 17.3 | 33.3 ± 5.8 | 40.5 ± 6.7 | 243.5 ± 12.4 | 28.2 ± 6.3 |
| Mix M: | 14.3 ± 6.6 | 272.3 ± 17.1 | 39.1 ± 4.0 | 25.0 ± 4.1 | 256.5 ± 29.9 | 44.0 ± 7.4 |
| Mix N: | 17.1 ± 2.7 | 285.7 ± 17.9 | 46.0 ± 3.1 | 15.9 ± 5.0 | 370.0 ± 29.9 | 56.2 ± 8.6 |
| Mix P: | 32.1 ± 2.3 | 213.1 ± 14.1 | 52.7 ± 5.8 | 20.0 ± 2.9 | 356.5 ± 24.6 | 40.9 ± 8.9 |

TABLE 2
Different Mixes being tested for 72 hour and 7 day time frames.

| Tested | % ± SD of control after 72 hrs | | | % ± SD of control after 7 days | | |
|---|---|---|---|---|---|---|
| mix | SP | RB | dead RB | SP | RB | dead RB |
| Mix A1: | 18.5 ± 3.0 | 492.3 ± 156 | 79.1 ± 9.6 | 10.0 ± 4.1 | 583.3 ± 37.3 | 72.7 ± 9.3 |
| Mix B: | 40.7 ± 3.7 | 338.5 ± 26.3 | 41.5 ± 4.1 | 13.3 ± 8.1 | 525.0 ± 11.6 | 43.1 ± 3.9 |
| Mix C: | 10.3 ± 4.3 | 480.6 ± 33.1 | 58.4 ± 5.4 | 11.2 ± 4.9 | 535.7 ± 69.9 | 41.2 ± 6.7 |
| Mix D: | 31.7 ± 2.4 | 247.6 ± 33.3 | 24.9 ± 7.5 | 35.4 ± 2.4 | 295.2 ± 19.5 | 27.6 ± 5.4 |
| Mix E: | 25.0 ± 8.7 | 300.0 ± 47.6 | 26.1 ± 3.9 | 16.7 ± 8.8 | 314.3 ± 11.4 | 28.6 ± 3.8 |
| Mix F: | 16.7 ± 8.9 | 319.0 ± 10.5 | 23.8 ± 7.6 | 12.5 ± 2.4 | 323.8 ± 22.3 | 34.1 ± 8.2 |
| Mix G: | 18.8 ± 4.7 | 408.1 ± 38.5 | 21.1 ± 4.7 | 14.6 ± 2.4 | 352.4 ± 29.9 | 29.6 ± 3.4 |
| Mix H: | 25.4 ± 3.8 | 300.0 ± 12.0 | 32.0 ± 4.0 | 25.4 ± 3.0 | 244.4 ± 22.3 | 33.2 ± 3.6 |
| Mix I: | 18.4 ± 6.4 | 403.7 ± 31.8 | 36.2 ± 4.3 | 19.1 ± 6.4 | 303.7 ± 18.6 | 39.3 ± 5.8 |
| Mix J: | 31.3 ± 1.9 | 265.4 ± 11.0 | 31.2 ± 7.2 | 34.4 ± 3.0 | 229.0 ± 16.6 | 53.2 ± 11.2 |
| Mix K: | 28.8 ± 1.0 | 280.8 ± 17.9 | 33.0 ± 8.1 | 25.0 ± 4.3 | 194.5 ± 18.5 | 47.6 ± 7.3 |
| Mix L: | 32.5 ± 6.6 | 323.1 ± 16.3 | 34.8 ± 4.1 | 31.3 ± 3.1 | 225.6 ± 17.1 | 39.1 ± 9.1 |
| Mix M: | 15.0 ± 4.1 | 265.4 ± 10.1 | 45.3 ± 4.7 | 12.5 ± 4.1 | 258.7 ± 12.6 | 46.1 ± 5.8 |
| Mix N: | 13.1 ± 4.8 | 334.6 ± 10.3 | 57.2 ± 4.0 | 12.5 ± 6.7 | 287.5 ± 15.3 | 49.2 ± 6.2 |
| Mix P: | 12.5 ± 6.7 | 396.2 ± 24.4 | 40.9 ± 8.9 | 6.3 ± 3.3 | 372.6 ± 21.2 | 57.4 ± 6.4 |

TABLE 3
Different Mixes being tested for Biofilm.

| Tested | Biofilm | Biofilm |
|---|---|---|
| mix | (prevention) | (cure) |
| Mix A1: | 32.8 ± 6.6 | 41.4 ± 3.2 |
| Mix B: | 62.1 ± 1.9 | 82.2 ± 7.0 |
| Mix C: | 43.8 ± 2.1 | 62.2 ± 9.7 |
| Mix D: | 43.4 ± 4.9 | 46.1 ± 2.3 |
| Mix E: | 77.7 ± 5.4 | 64.8 ± 4.8 |
| Mix F: | 69.2 ± 5.9 | 58.4 ± 9.9 |
| Mix G: | 46.8 ± 8.4 | 56.2 ± 5.5 |
| Mix H: | 44.4 ± 6.1 | 53.0 ± 4.9 |
| Mix I: | 54.0 ± 8.9 | 47.8 ± 4.3 |
| Mix J: | 52.8 ± 7.1 | 58.1 ± 3.6 |
| Mix K: | 41.3 ± 5.3 | 62.2 ± 8.8 |
| Mix L: | 51.9 ± 4.1 | 70.1 ± 4.8 |
| Mix M: | 38.4 ± 9.0 | 39.8 ± 4.1 |
| Mix N: | 15.6 ± 4.8 | 33.4 ± 6.2 |
| Mix P: | 50.2 ± 6.8 | 55.2 ± 8.4 |

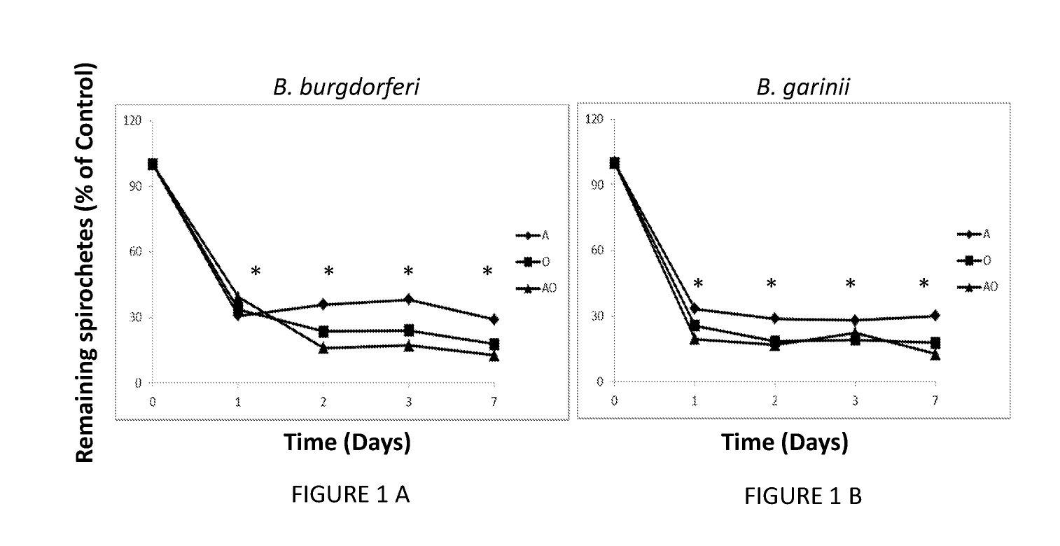

The various Figures are discussed in detail below. FIG. 1A shows susceptibility of the spirochetes of B. burgdorferi (left panel) and FIG. 1 B B. garinii (right panel) to the most effective concentration of the chosen three the most effective mixtures composed of naturally derived substances selected after screening of different compounds (Vitamins and Phytobiologicals) at different concentration that were evaluated up to 7 days by dark-field microscope. Results have shown that all mixtures significantly decrease amount of spiral forms of both tested Borrelia species about 70-75%.

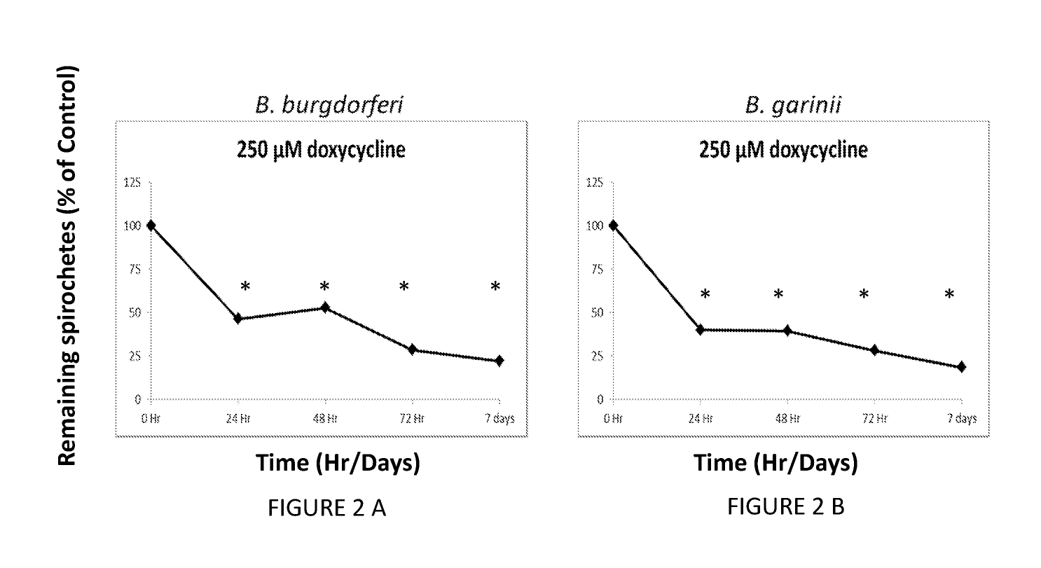

FIG. 2 A shows susceptibility of the spirochetes of B. burgdorferi (left panel) and FIG. 2 B B. garinii (right panel) of to the commonly tested concentration of antibiotic doxycycline that is use as a frontline treatment in Lyme disease evaluated up to 7 days by dark-field microscopy. Results have shown that doxycycline significantly decreases amount of spiral forms of both tested Borrelia species about 60-75%.

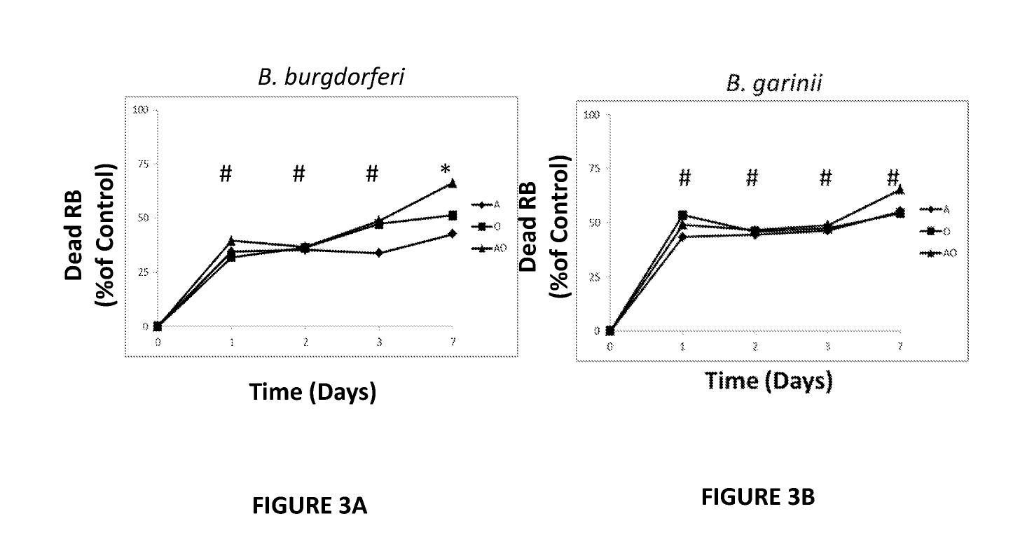

FIG. 3 A shows susceptibility of the rounded forms of B. burgdorferi (left panel) and FIG. 3 B B. garinii (right panel) to the most effective concentration of the chosen three most effective mixtures composed of naturally derived substances selected after screening of different compounds (vitamins and Phytobiologicals) at different concentration that were evaluated up to 7 days by fluorescent microscope using SYTO® 9 green-fluorescent stain (live organisms) and propidium iodide red-fluorescent stain (dead organisms). Results have shown that all mixtures significantly increase dead of rounded forms about 50-60% (A panel) and generate rounded forms formation about 2-3 times in both tested Borrelia species (B panel).

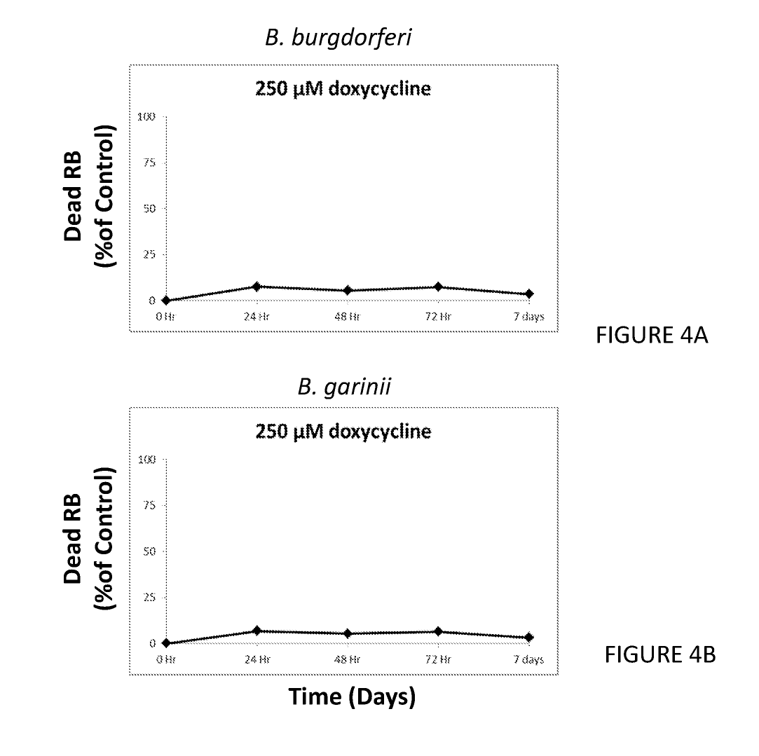

FIG. 4 A shows susceptibility of the rounded forms of B. burgdorferi (upper panel) and FIG. 4 B B. garinii (lower panel) to the most effective concentration of antibiotic doxycycline used as a frontline treatment of Lyme disease evaluated up to 7 days by fluorescent microscope using SYTO® 9 green-fluorescent stain (live organisms) and propidium iodide red-fluorescent stain (dead organisms). Results have shown that doxycycline increases dead of rounded forms about 5-10% (A panel) and generate rounded forms formation about 2 times in both tested Borrelia species (B panel).

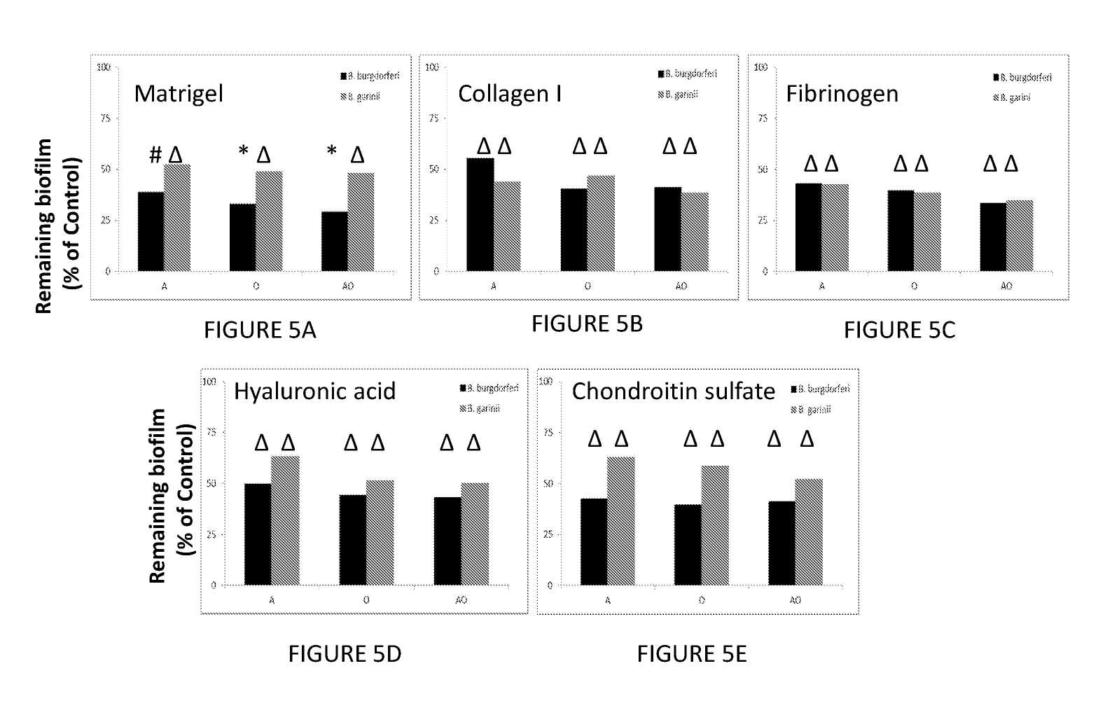

FIGS. 5 A, B, C, D and E shows quantitative analysis of remaining biofilm of B. burgdorferi and B. garinii cultured on 5 different extracellular matrix proteins such as: matrigel, collagen type I, fibrinogen, hyaluronic acid, and chondroitin sulfate to the most effective concentration of the chosen three most effective mixtures composed of naturally derived substances selected after screening of different compounds (vitamins and Phytobiologicals) at different concentration that were measured 3 days of post-treatment by crystal violet staining technique. Results have shown that all mixtures significantly eradicate biofilm of both tested Borrelia species about 40-55% regardless of extracellular matrix matrices used.

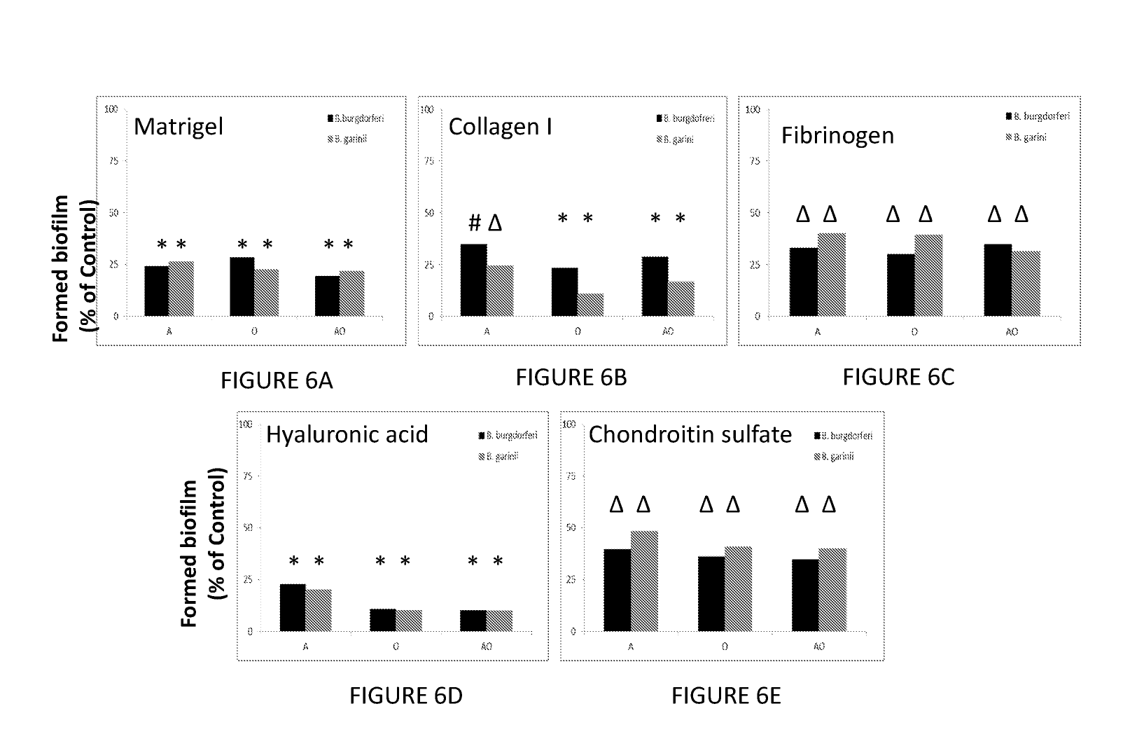

FIGS. 6 A, B, C, D and E shows quantitative analysis of formed biofilm of B. burgdorferi and B. garinii on 5 different extracellular matrix proteins such as: matrigel, collagen type I, fibrinogen, hyaluronic acid, and chondroitin sulfate to the most effective concentration of the chosen three most effective mixtures composed of naturally derived substances selected after screening of different compounds (vitamins and Phytobiologicals) at different concentration that were measured after 7 days by crystal violet staining technique. Results have shown that all mixtures significantly prevented biofilm formation of both tested Borrelia species about 50-65% regardless of extracellular matrix matrices used.

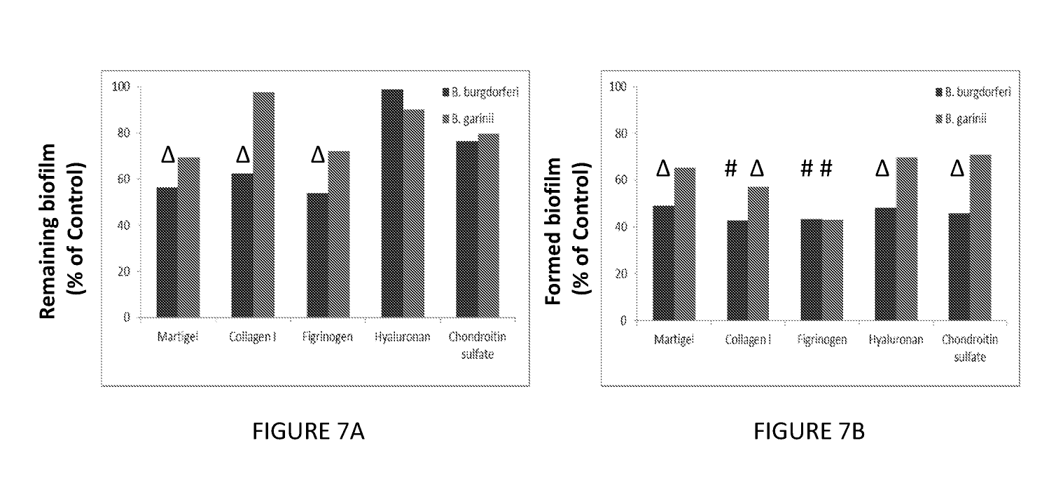

FIG. 7 A shows quantitative evaluation of biofilm of B. burgdorferi and FIG. 7 B shows B. garinii cultured on 5 different extracellular matrix proteins such as: matrigel, collagen type I, fibrinogen, hyaluronic acid, and chondroitin sulfate (curable effect—left panel) and (preventive effect—right panel) to the most effective concentration of antibiotic doxycycline use as a frontline treatment in Lyme disease by crystal violet staining technique. Results have shown that doxycycline eradicates biofilm of both tested Borrelia species ˜5-40% and prevents form biofilm formation of both tested Borrelia species about 40-60% depends of type of protein matrices used.

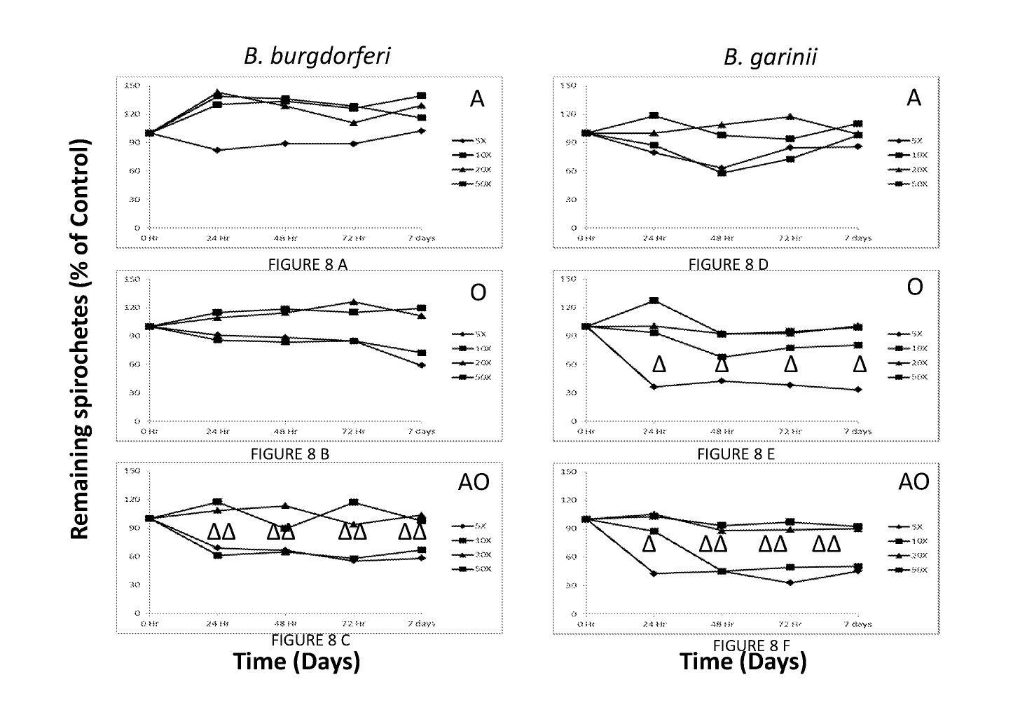

FIGS. 8 A, B, C, D and E shows susceptibility of the spirochetes of B. burgdorferi (left panel) and B. garinii (right panel) to the different concentrations of the chosen three most effective mixtures composed of naturally derived substances selected after screening of different compounds (Vitamins and Phytobiologicals) at different concentration that were evaluated up to 7 days by dark-field microscope. Results have shown that mixture AO diluted up to 10× significantly decreases amount of spiral forms of both tested Borrelia species about 40-45%.

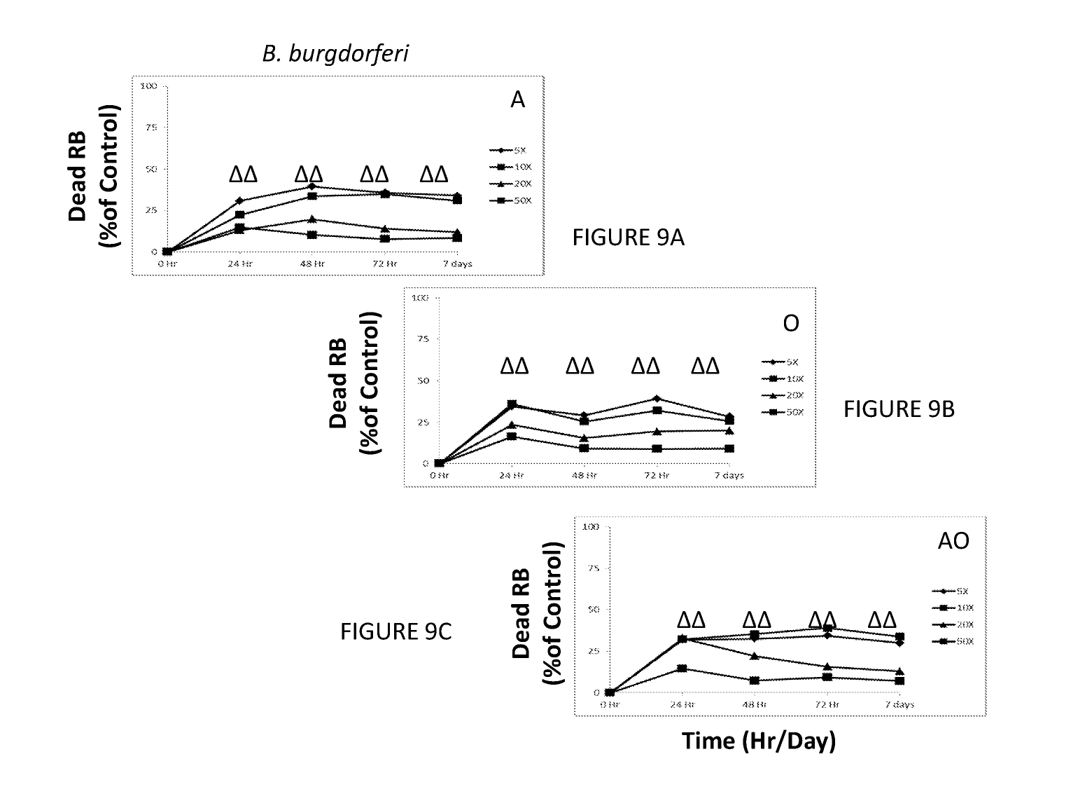

FIGS. 9 A, B and C shows susceptibility of the rounded forms of B. burgdorferi to the different concentrations of the chosen three most effective mixtures composed of naturally derived substances selected after screening of different compounds (vitamins and Phytobiologicals) at different concentration that were evaluated up to 7 days by fluorescent microscope using SYTO® 9 green-fluorescent stain (live organisms) and propidium iodide red-fluorescent stain (dead organisms). Results have shown that all mixtures diluted up to 10× significantly increase dead of rounded forms about 30-40% (A panel) and generate rounded forms formation about 1.8 times (B panel).

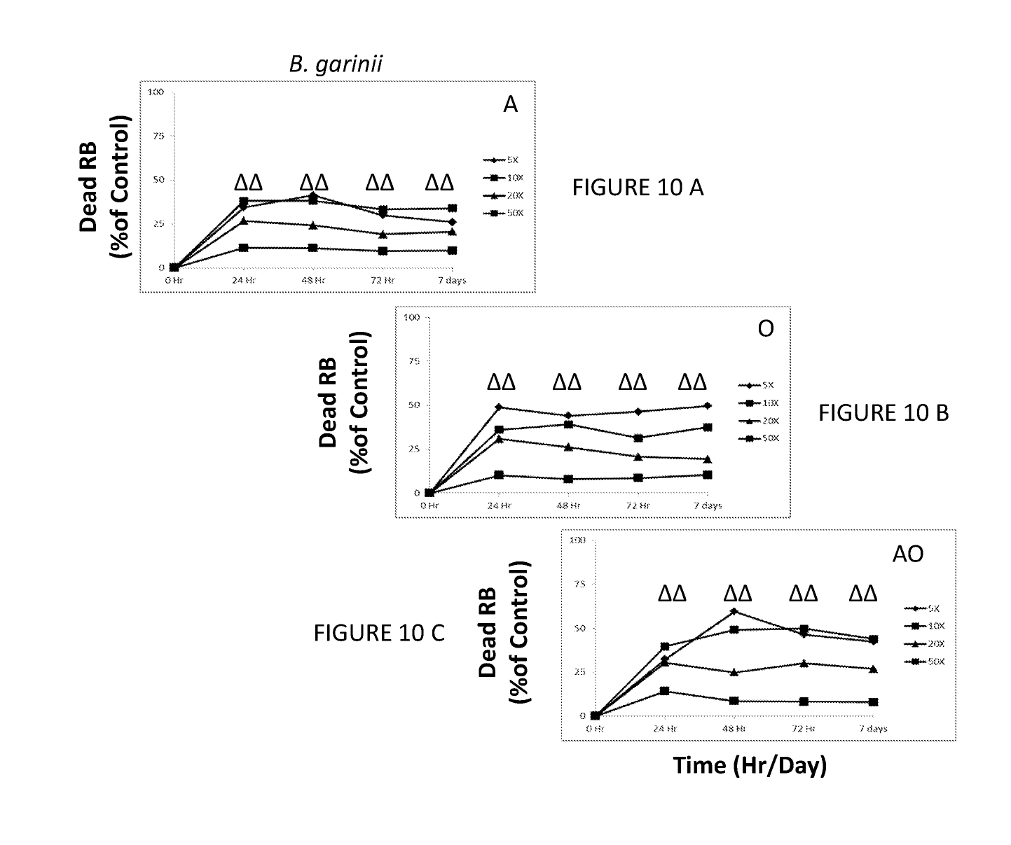

FIGS. 10 A, B and C shows susceptibility of the rounded forms of B. garinii to the different concentrations of the chosen three most effective mixtures composed of naturally derived substances selected after screening of different compounds (Vitamins and Phytobiologicals) at different concentration that were evaluated up to 7 days by fluorescent microscope using SYTO® 9 green-fluorescent stain (live organisms) and propidium iodide red-fluorescent stain (dead organisms). Results have shown that all mixtures diluted up to 10× significantly increase dead of rounded forms about 40-50% (A panel) and generate rounded forms formation about 1.8 times (B panel).

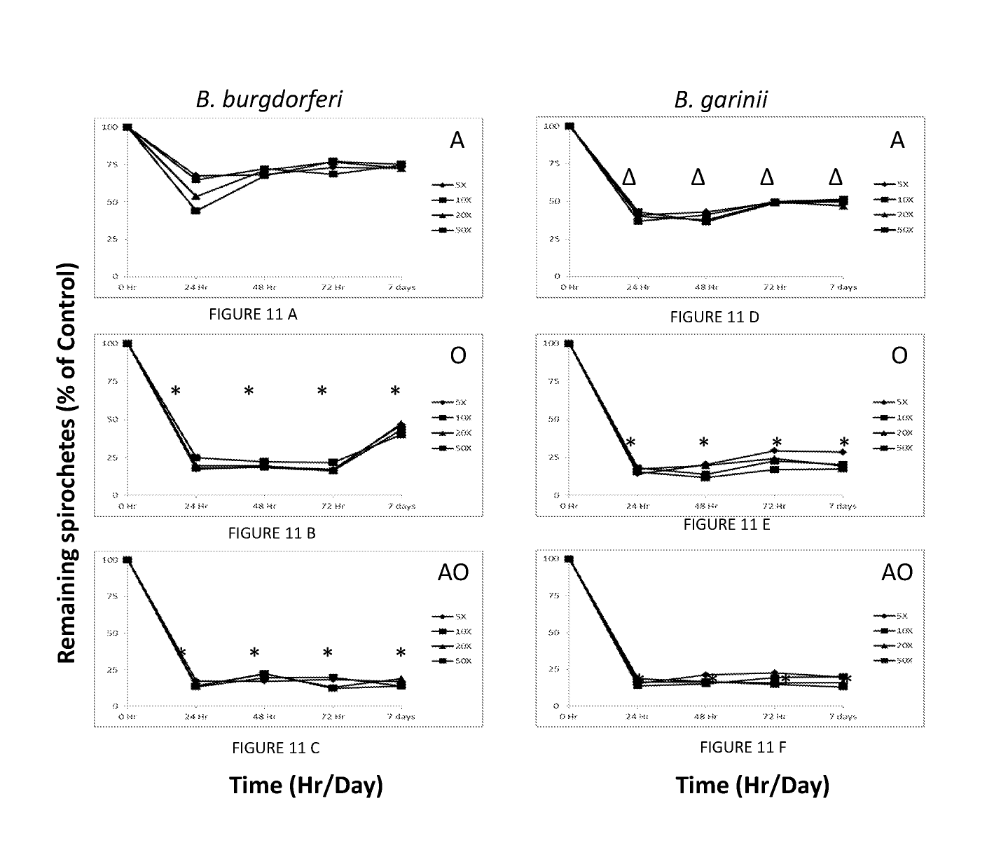

FIGS. 11 A, B, C, D and E shows Susceptibility of the spirochetes of B. burgdorferi (left panel) and B. garinii (right panel) treated with different concentrations of the chosen three most effective mixtures composed of naturally derived substances selected after screening of different compounds (vitamins and Phytobiologicals) at different concentration that were evaluated for up to 4 weeks and evaluated by dark-field microscope. Results have shown that mixtures O and AO at the most effective concentration, regardless of sub-dose used, significantly decrease amount of spiral forms of both tested Borrelia species about 60-70%.

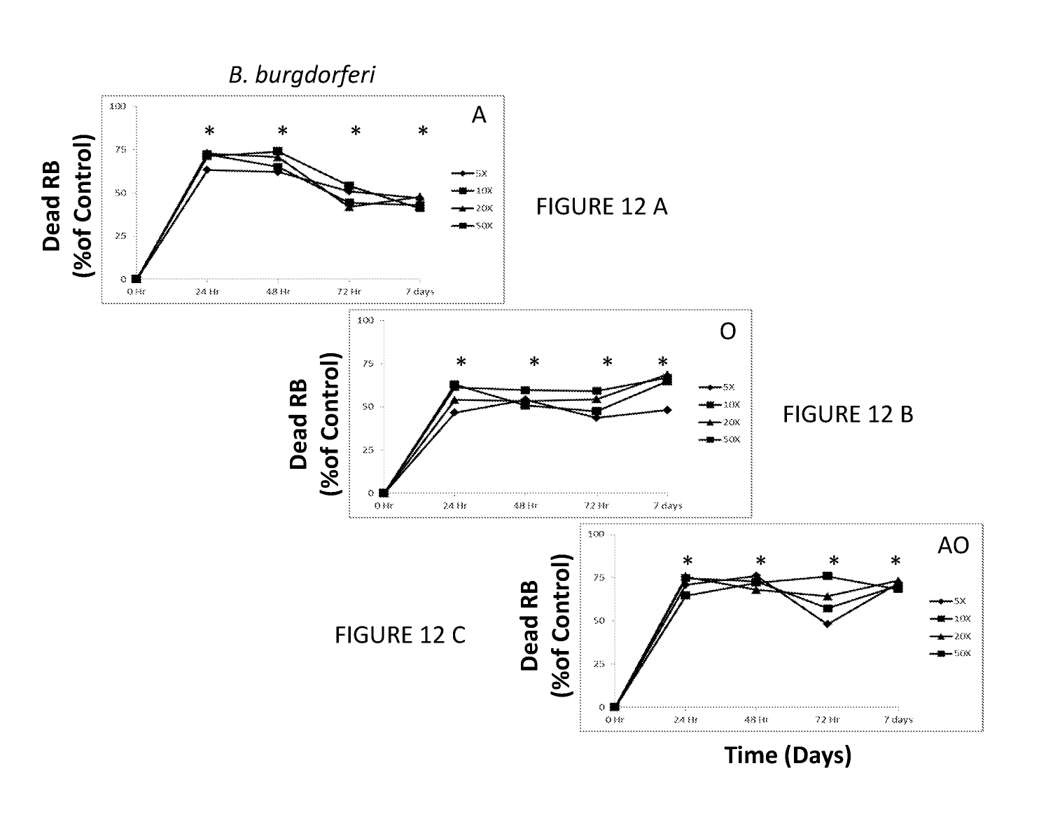

FIGS. 12 A, B and C shows susceptibility of the rounded forms of B. burgdorferi to the different concentrations of the chosen three most effective mixtures composed of naturally derived substances selected after screening of different compounds (vitamins and Phytobiologicals) at different concentration that were evaluated up to 4 weeks by fluorescent microscope using SYTO® 9 green-fluorescent stain (live organisms) and propidium iodide red-fluorescent stain (dead organisms). Results have shown that all mixtures at the most effective concentration, regardless of sub-dose used, significantly increase dead of rounded forms about 60-65% (A panel) and generate rounded forms formation about 2-3 times (B panel).

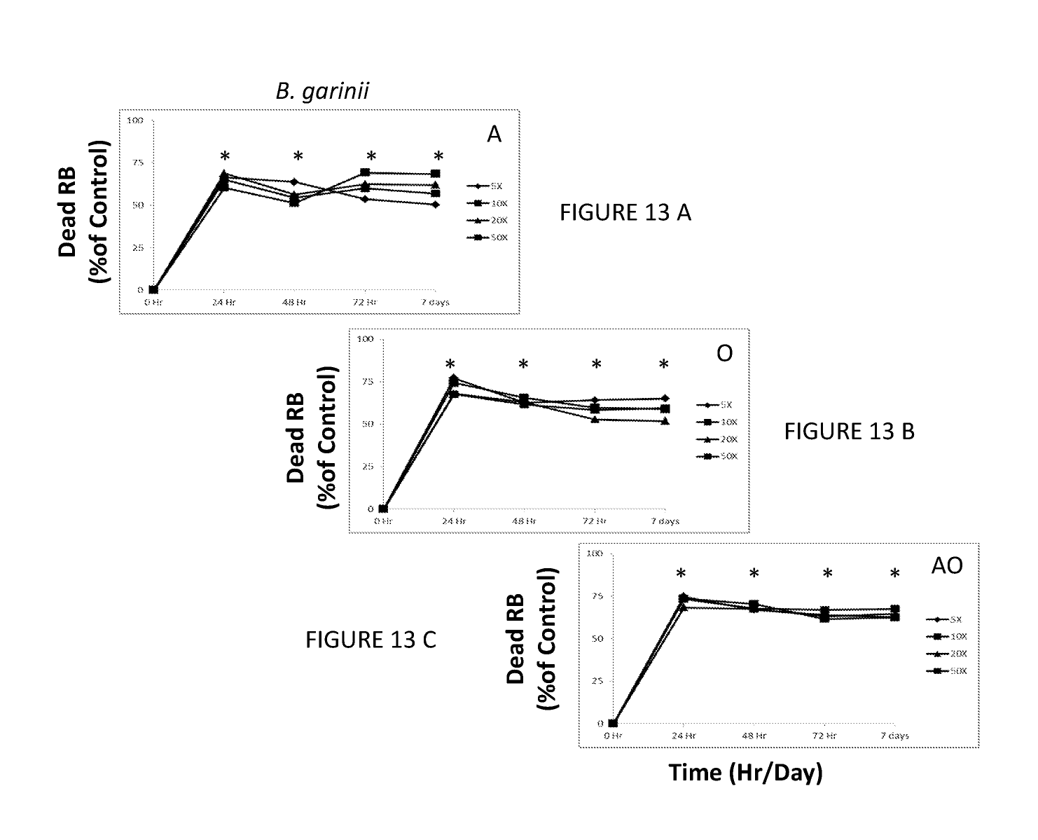

FIGS. 13 A, B and C shows susceptibility of the rounded forms of B. garinii to the different concentrations of the chosen three most effective mixtures composed of naturally derived substances selected after screening of different compounds (vitamins and Phytobiologicals) at different concentration that were evaluated up to 4 weeks by fluorescent microscope using SYTO® 9 green-fluorescent stain (live organisms) and propidium iodide red-fluorescent stain (dead organisms). Results have shown that all mixtures at the most effective concentration, regardless of sub-dose used, significantly increase dead of rounded forms about 60-70% (A panel) and generate rounded forms formation about 1.8-2 times (B panel).

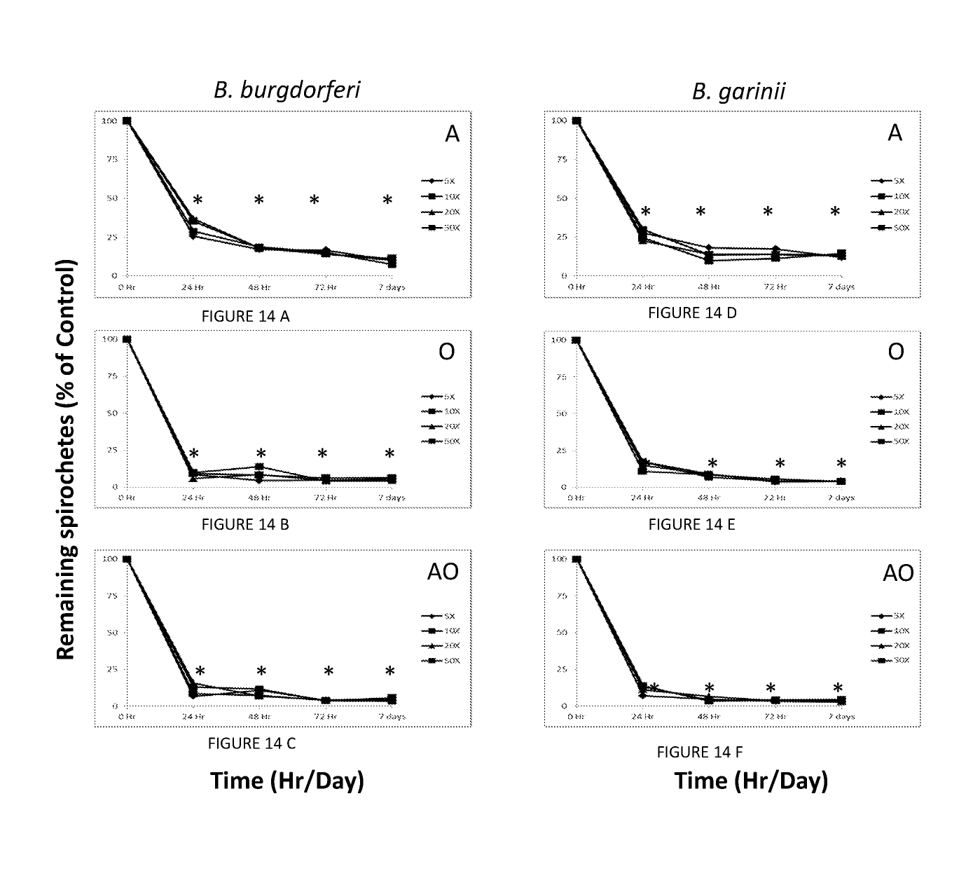

FIGS. 14 A, B, C, D and E shows susceptibility of the spirochetes of B. burgdorferi (left panel) and B. garinii (right panel) treated with different concentrations of the chosen three most effective mixtures composed of naturally derived substances selected after screening of different compounds (vitamins and Phytobiologicals) at different concentration that were evaluated up to 5 months and evaluated by dark-field microscope. Results have shown that all mixtures at the most effective concentration regardless of sub-dose used significantly decrease amount of spiral forms of both tested Borrelia species about 70-90%.

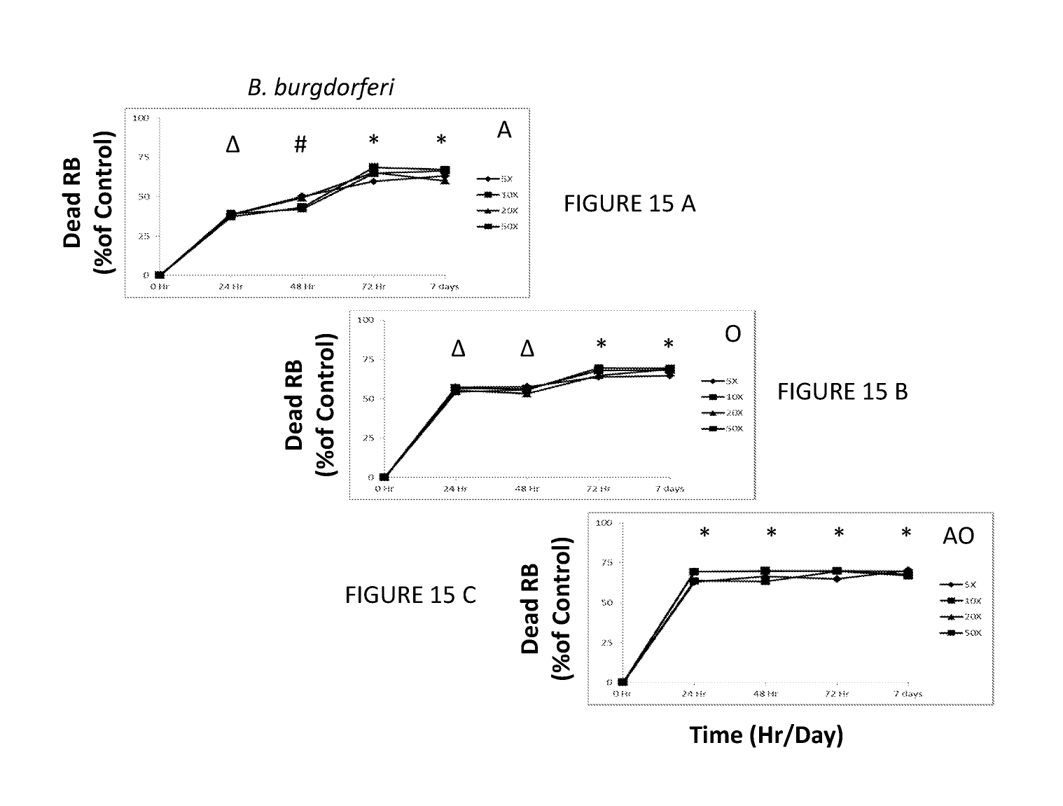

FIGS. 15 A, B and C shows susceptibility of the rounded forms of B. burgdorferi to the different concentrations of the chosen three most effective mixtures composed of naturally derived substances selected after screening of different compounds (vitamins and Phytobiologicals) at different concentration that were evaluated up 5 months by fluorescent microscope using SYTO® 9 green-fluorescent stain (live organisms) and propidium iodide red-fluorescent stain (dead organisms). Results have shown that all mixtures at the most effective concentration, regardless of sub-dose used, significantly increase dead of rounded forms about 60-70% (A panel) and generate rounded forms formation about 2-3 times (B panel).

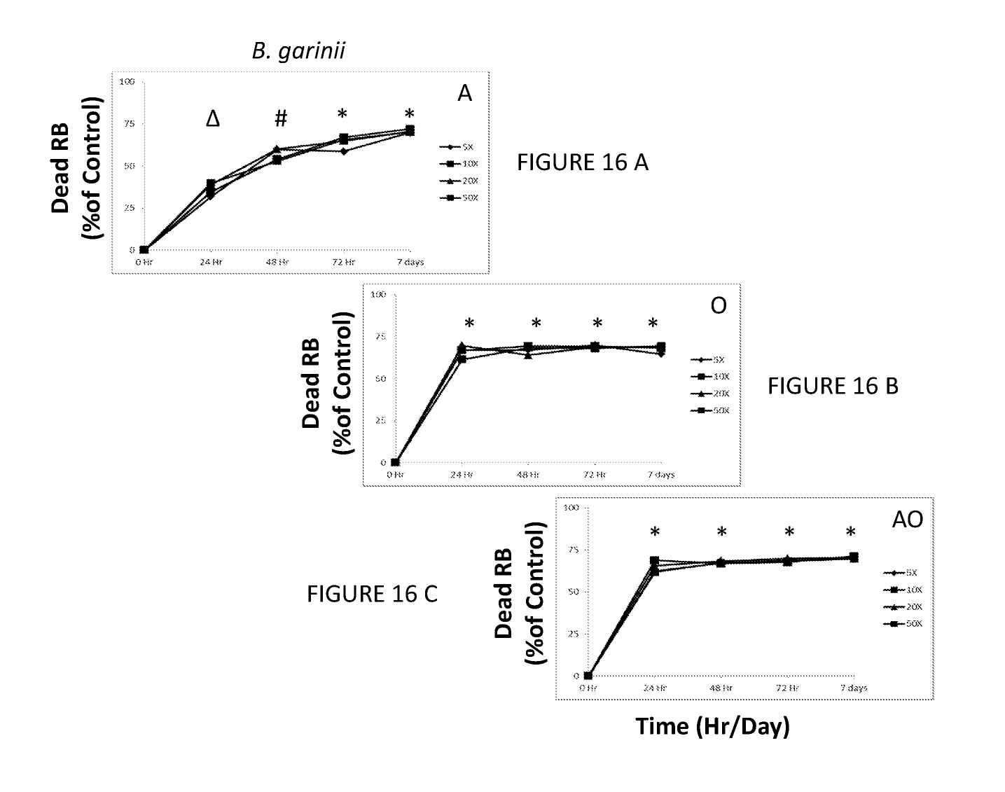

FIGS. 16 A, B and C shows susceptibility of the rounded forms of B. garinii to the different concentrations of the chosen three most effective mixtures composed of naturally derived substances selected after screening of different compounds (vitamins and Phytobiologicals) at different concentration that were evaluated up 5 months by fluorescent microscope using SYTO® 9 green-fluorescent stain (live organisms) and propidium iodide red-fluorescent stain (dead organisms). Results have shown that all mixtures at the most effective concentration, regardless of sub-dose used, significantly increase dead of rounded forms about 60-70% (A panel) and generate rounded forms formation about 2-3 times (B panel). All the above figures show that a prevention of Lyme disease is being performed by these Mixes from the diseases to progress from one stage to another. The method making a mixture at a specific concentration and using the mixture for a specific duration to treat and prevent progression of the disease from the acute stage to the chronic stage, wherein the prevent the progression of the advancement of the disease from a spirochete stage to a round bodied and to a formation of biofilms in human and other mammals is described with results in detail in all figures. Drug formulations suitable for these administration routes can be produced by adding one or more pharmacologically acceptable carriers to the agent and then treating the mixture through a routine process known to those skilled in the art. The mode of administration includes, but not limited to, are non-invasive perioral, topical (example transdermal), enteral, transmucosal, targeted delivery, sustained release delivery, delayed release, pulsed release and parenteral methods. Various combinations of these treatments may also be combined. Perioral administration may be administered both in liquid and dry state. The drug formulations for oral consumption for example should have the composition for Mix AO as follows: Vitamin D3 1000 IU-50,000 IU, Cis-2-Decenoic acid 80 mg-8000 mg, Kelp (Iodine) 150 mcg-1000 mcg, Monolaurin 50 mg-5000 mg, Luteolin 50 mg-2000 mg, Rosmarinic acid 50 mg-3000 mg, Vitamin B complex 1×RDA—100×RDA, Vitamin C 100 mg-20,000 mg, Baicalein 50 mg-5000 mg.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition as an active ingredient. Subject compositions may also be administered as a bolus, electuary, or paste.

When an oral solid drug product is prepared, combination of Mix A to P, AO is mixed with an excipient (and, if necessary, one or more additives such as a binder, a disintegrant, a lubricant, a coloring agent, a sweetening agent, and a flavoring agent), and the resultant mixture is processed through a routine method, to thereby produce an oral solid drug product such as tablets, coated tablets, granules, powder, or capsules. Additives may be those generally employed in the art. Examples of the excipient include lactate, sucrose, sodium chloride, glucose, starch, calcium carbonate, kaolin, microcrystalline cellulose, and silicic acid; examples of the binder include water, ethanol, propanol, simple syrup, glucose solution, starch solution, liquefied gelatin, carboxymethyl cellulose, hydroxypropyl cellulose, hydroxypropyl starch, methyl cellulose, ethyl cellulose, shellac, calcium phosphate, and polyvinyl pyrrolidone; examples of the disintegrant include dried starch, sodium arginate, powdered agar, sodium hydrogencarbonate, calcium carbonate, sodium lauryl sulfate, monoglyceryl stearate, and lactose; examples of the lubricant include purified talc, stearic acid salts, borax, and polyethylene glycol; and examples of the sweetening agent include sucrose, orange peel, citric acid, and tartaric acid.

When a liquid drug product for oral administration is prepared, Mix A to P, AO is mixed with an additive such as a sweetening agent, a buffer, a stabilizer, or a flavoring agent, and the resultant mixture is processed through a routine method, to thereby produce an orally administered liquid drug product such as an internal solution medicine, syrup, or elixir. Examples of the sweetening agent include vanillin; examples of the buffer include sodium citrate; and examples of the stabilizer include tragacanth, acacia, and gelatin.

For purposes of transdermal (e.g., topical) administration, dilute sterile, aqueous or partially aqueous solutions (usually in about 0.1% to 5% concentration), otherwise similar to the above parenteral solutions, may be prepared.

Formulations for rectal or vaginal administration may be presented as a suppository, which may be prepared by mixing a subject composition with one or more suitable non-irritating carriers comprising, for example, cocoa butter, polyethylene glycol, a suppository wax, or a salicylate, and which is solid at room temperature, but liquid at body temperature and, therefore, will melt in the appropriate body cavity and release the encapsulated compound(s) and composition(s). Formulations which are suitable for vaginal administration also include pessaries, tampons, creams, gels, pastes, foams, or spray formulations containing such carriers as are known in the art to be appropriate.

A targeted release portion can be added to the extended release system by means of either applying an immediate release layer on top of the extended release core; using coating or compression processes or in a multiple unit system such as a capsule containing extended and immediate release beads.

When used with respect to a pharmaceutical composition or other material, the term “sustained release” is art-recognized. For example, a therapeutic composition which releases a substance over time may exhibit sustained release characteristics, in contrast to a bolus type administration in which the entire amount of the substance is made biologically available at one time. For example, in particular embodiments, upon contact with body fluids including blood, spinal fluid, mucus secretions, lymph or the like, one or more of the pharmaceutically acceptable excipients may undergo gradual or delayed degradation (e.g., through hydrolysis) with concomitant release of any material incorporated therein, e.g., an therapeutic and/or biologically active salt and/or composition, for a sustained or extended period (as compared to the release from a bolus). This release may result in prolonged delivery of therapeutically effective amounts of any of the therapeutic agents disclosed herein.

Current efforts in the area of drug delivery include the development of targeted delivery in which the drug is only active in the target area of the body (for example, in cancerous tissues) and sustained release formulations in which the drug is released over a period of time in a controlled manner from a formulation. Types of sustained release formulations include liposomes, drug loaded biodegradable microspheres and drug polymer conjugates.

Delayed release dosage formulations are created by coating a solid dosage form with a film of a polymer which is insoluble in the acid environment of the stomach, but soluble in the neutral environment of the small intestines. The delayed release dosage units can be prepared, for example, by coating a drug or a drug-containing composition with a selected coating material. The drug-containing composition may be a tablet for incorporation into a capsule, a tablet for use as an inner core in a “coated core” dosage form, or a plurality of drug-containing beads, particles or granules, for incorporation into either a tablet or capsule. Preferred coating materials include bio-erodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Alternatively, a delayed release tablet may be formulated by dispersing tire drug within a matrix of a suitable material such as a hydrophilic polymer or a fatty compound. Suitable hydrophilic polymers include, but are not limited to, polymers or copolymers of cellulose, cellulose ester, acrylic acid, methacrylic acid, methyl acrylate, ethyl acrylate, and vinyl or enzymatically degradable polymers or copolymers as described above. These hydrophilic polymers are particularly useful for providing a delayed release matrix. Fatty compounds for use as a matrix material include, but are not limited to, waxes (e.g. carnauba wax) and glycerol tristearate. Once the active ingredient is mixed with the matrix material, the mixture can be compressed into tablets.

A pulsed release-dosage is one that mimics a multiple dosing profile without repeated dosing and typically allows at least a twofold reduction in dosing frequency as compared to the drug presented as a conventional dosage form (e.g., as a solution or prompt drug-releasing, conventional solid dosage form). A pulsed release profile is characterized by a time period of no release (lag time) or reduced release followed by rapid drug release.

The phrases “parenteral administration” and “administered parenterally” as used herein refer to modes of administration other than enteral and topical administration, such as injections, and include without limitation intravenous, intramuscular, intrapleural, intravascular, intrapericardial, intra-arterial, intrathecal, intracapsular, intraorbital, intracardiac, intradennal, intraperitoneal, transtracheal, subcutaneous, subcuticular, intra-articular, subcapsular, subarachnoid, intraspinal and intrastemal injection and infusion.

Certain pharmaceutical compositions disclosed herein suitable for parenteral administration comprise one or more subject compositions in combination with one or more pharmaceutically acceptable sterile, isotonic, aqueous, or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic within the blood of the intended recipient or suspending or thickening agents.

When an injection product is prepared, Mix A to P, AO is mixed with an additive such as a pH regulator, a buffer, a stabilizer, an isotonicity agent, or a local anesthetic, and the resultant mixture is processed through a routine method, to thereby produce an injection for subcutaneous injection, intramuscular injection, or intravenous injection. Examples of the pH regulator or buffer include sodium citrate, sodium acetate, and sodium phosphate; examples of the stabilizer include sodium pyrosulfite, EDTA, thioglycollic acid, and thiolactic acid; examples of the local anesthetic include procaine hydrochloride and lidocaine hydrochloride; and examples of the isotonicity agent include sodium chloride and glucose.

The phrase “pharmaceutically acceptable” is art-recognized. In certain embodiments, the term includes compositions, polymers and other materials and/or dosage forms which are within the scope of sound medical judgment, suitable for use in contact with the tissues of mammals, human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the pharmaceutical compositions described herein are formulated in a manner such that said compositions will be delivered to a mammal in a therapeutically effective amount, as part of a prophylactic, preventive or therapeutic treatment.

In certain embodiments, the dosage of the Mix A to P, AO compositions, which may be referred as therapeutic composition provided herein may be determined by reference to the plasma concentrations of the therapeutic composition or other encapsulated materials. For example, the blood samples may be tested for the presence or absence of Bacterial infection.

The therapeutic compositions provided by this application may be administered to a subject in need of treatment by a variety of conventional routes of administration, including orally, topically, parenterally, e.g., intravenously, subcutaneously or intramedullary. Further, the therapeutic compositions may be administered intranasally, as a rectal suppository, or using a “flash” formulation, i.e., allowing the medication to dissolve in the mouth without the need to use water. Furthermore, the compositions may be administered to a subject in need of treatment by controlled release dosage forms, site specific drug delivery, transdermal drug delivery, patch (active/passive) mediated drug delivery, by stereotactic injection, or in nanoparticles.

Expressed in terms of concentration, an active ingredient can be present in the therapeutic compositions of the present invention for localized use about the cutis, intranasally, pharyngolaryngeally, bronchially, intravaginally, rectally, or ocularly.

For use as aerosols, the active ingredients can be packaged in a pressurized aerosol container together with a gaseous or liquefied propellant, for example, dichlorodifluoromethane, carbon dioxide, nitrogen, propane, and the like, with the usual adjuvants such as cosolvents and wetting agents, as may be necessary or desirable.

The most common routes of administration also include the preferred transmucosal (nasal, buccal/sublingual, vaginal, ocular and rectal) and inhalation routes.

In addition, in certain embodiments, subject compositions of the present application maybe lyophilized or subjected to another appropriate drying technique such as spray drying. The subject compositions may be administered once, or may be divided into a number of smaller doses to be administered at varying intervals of time, depending in part on the release rate of the compositions and the desired dosage.

Formulations useful in the methods provided herein include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of a subject composition which may be combined with a carrier material to produce a single dose may vary depending upon the subject being treated, and the particular mode of administration.

The therapeutically acceptable amount described herein may be administered in inhalant or aerosol formulations. The inhalant or aerosol formulations may comprise one or more agents, such as adjuvants, diagnostic agents, imaging agents, or therapeutic agents useful in inhalation therapy. The final aerosol formulation may for example contain 0.005-90% w/w, for instance 0.005-50%, 0.005-5% w/w, or 0.01-1.0% w/w, of medicament relative to the total weight of the formulation.

Examples of suitable aqueous and non-aqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

The therapeutic acceptable dosage may be combined with other drugs and may be treated as a combination drug. In one embodiment instructions teaching the use of the Mix A to P, AO response assay kit according to the various methods and approaches described herein are provided. Such kits may also include information, such as scientific literature references, package insert materials, clinical trial results, and/or summaries of these and the like, which indicate or establish the activities and/or advantages of the agent. Kits described herein can be provided, marketed and/or promoted to health providers, including physicians, nurses, pharmacists, formulary officials, and the like.

In addition, it will be appreciated that the various Mix's, dosage and methods of treatment disclosed herein may be embodied using means for achieving the various combinations of therapeutic dosage and delivery methods to treat a specific disease. Accordingly, the specification and drawings are to be regarded in an illustrative rather than a restrictive sense.

What is claimed is:

1. A composition, comprising:
a mixture having a specific concentration of at least one of a Vitamin, a Small chain fatty acid signaling molecule, an Essential Amino Acid, a Lauric acid derivative, a Flavonoid, a Phenol, a Plant Extract, and a Mineral for treating a Lyme disease.

2. The composition of claim 1, wherein the vitamin is at least one of a Vitamin C, Vitamin B complex and Vitamin D3, the small fatty acid signaling molecule is Cis-2-decenoic acid, the essential amino acid is at least one of a L-Lysin, Aminocaproic acid and Tranexamic acid, the flavonoid is at least one of a Luteolin, Apigenin, Baicalein, Fisetin, Kaempherol, Myricetin and Quercetin 3D, the plant extract is at least one of a Oleuropein, Teasel Root Extract, Oregano Oil, Kelp, Monolaurin, Nordihydroguaiaretic acid, Fucoinad, Olein, Rosmarinic acid, Morin, Aronia, Rottlerin, Malvidin, Grape seed extract (OPC), Piceatannol the phenol is at least one of a Ellagic acid and E-Viniferin and the mineral is an Kelp (Iodine).

3. The composition of claim 1, wherein the mixture is at least one of a Mix A, Mix A1, Mix B, Mix C, Mix D, Mix E, Mix F, Mix G, Mix H, Mix I, Mix J, Mix K, Mix L, Mix M, Mix N, Mix O, Mix AO and Mix P.

4. The composition of claim 1, wherein the mixture is a Mix A, wherein the Mix A consists of a Vitamin D3, Cis-2-Decenoic acid, Kelp (Iodine), Monolaurin, Luteolin and Rosmarinic acid.

5. The composition of claim 1, wherein the mixture is a Mix O, wherein the Mix O consists of a Vitamin B complex, Vitamin C, Kelp (Iodine), Monolaurin, Rosmarinic acid and Baicalein.

6. The composition of claim 1, wherein the mixture is a Mix AO, wherein the Mix AO consists of a Vitamin D3, Cis-2-decenoic acid, Kelp (Iodine), Monolaurin, Luteolin, Rosmarinic acid, Vitamin B complex, Vitamin C and Baicalein.

7. The composition of claim 1, wherein the Lyme disease is caused by at least one of a B. burgdorferi and B. garinii in a different form, wherein the different form is spirochetes, rounded bodies and biofilm.

8. A method, comprising:
making a mixture at a specific concentration using at least one of a vitamin, a small chain fatty acid signaling molecule, an essential amino acid, a Lauric acid derivative, a flavonoid, a phenol, a plant extract, and a mineral; and
treating of a Lyme disease caused by specific bacteria using the mixture and
preventing progression of the disease from the acute stage to the chronic stage, wherein the preventing progression of the Lyme disease is the advancement of the Lyme disease from spirochete stage to a rounded bodies and further to the formation of a biofilm.

9. The method of claim 8, wherein the specific bacteria is at least one of a B. burgdorferi and B. garinii in a different morphological form.

10. The method of claim 9, wherein the different forms are one of a spirochetes, rounded bodies and biofilm.

11. The method of claim 8, wherein the mixture is at least one of a Mix A, Mix A1, Mix B, Mix C, Mix D, Mix E, Mix F, Mix G, Mix H, Mix I, Mix J, Mix K, Mix L, Mix M, Mix N, Mix O, Mix AO and Mix P.

12. The composition of claim 8, wherein the treating of the Lyme disease may be performed using at least one of a non-invasive perioral, topical (example transdermal), enteral, transmucosal, targeted delivery, sustained release delivery, delayed release, pulsed release, parenteral methods
and a combination thereof.

13. The method of claim 8, wherein the mixture is a Mix AO, wherein the Mix AO consists of a Vitamin D3—0.5 nM-1.5 nM, Cis-2-decenoic acid—200-300 μg/ml, Kelp (Iodine)—1-10 μg/ml, Monolaurin—500-1500 μg/ml, Luteolin—50-150 μg/ml, Rosmarinic acid—150-300 μg/ml, Vitamin B complex—0.5-2.0 mg/ml, Vitamin C—0.1-0.3 mM, Baicalein—500-1500 μg/ml.

14. A method, comprising:
making a mixture at a specific concentration and using the mixture for a specific duration to treat and prevent progression of the disease from the acute stage to the chronic stage, wherein the prevent the progression of the advancement of the disease from a spirochete stage to a rounded bodies and to a formation of biofilms in human and other mammals; and
delivering the mixture in a specific form for treating and preventing for a specific duration.

15. The method of claim 14, wherein the specific duration is a short term and long term duration.

16. The method of claim 14, wherein the mixture is at least one of a Mix A, Mix A1, Mix B, Mix C, Mix D, Mix E, Mix F, Mix G, Mix H, Mix I, Mix J, Mix K, Mix L, Mix M, Mix N, Mix O, Mix AO and Mix P.

17. The composition of claim 14, wherein the specific form is at least one of a tablet, syrup, injection, intravenous and combination thereof.

18. The method of claim 14, wherein the mixture is a Mix AO, wherein the mixture is a Mix AO, wherein the specific concentration of the Mix AO consists of a Vitamin D3—0.5 nM-1.5 nM, Cis-2-decenoic acid—200-300 μg/ml, Kelp (Iodine)—1-10 μg/ml, Monolaurin—500-1500 μg/ml, Luteolin—50-150 μg/ml, Rosmarinic acid—150-300 μg/ml, Vitamin B complex—0.5-2.0 mg/ml, Vitamin C—0.1-0.3 mM, Baicalein—500-1500 μg/ml.

19. The method of claim 14, wherein the mixture is a Mix A and the specific concentration of Mix A consists of a Vitamin D3—0.5-1.5 nM, Cis-2-decenoic acid—150-300 μg/ml, Kelp (Iodine)—1-10 μg/ml, Monolaurin—500-1500 μg/ml, Luteolin—50-150 μg/ml, Rosmarinic acid—200-300 μg/ml.

20. The method of claim 14, wherein the mixture is Mix O, wherein the specific concentration in Mix O consists of a Vitamin B complex—0.5-1.5 mg/ml, Vitamin C—0.1-0.3 mM, Kelp (Iodine)—1-10 μg/ml, Monolaurin—500-1500 μg/ml, Rosmarinic acid—200-300 μg/ml, Baicalein—500-1500 μg/ml.
